(19) United States
(12) Patent Application Publication
(10) Pub. No.: US 2005/0020561 A1
(43) Pub. Date: Jan. 27, 2005

(54) Process for the preparation of cefpodoxime acid

(76) Inventors: Yatendra Kumar, Haryana (IN); Neera Tewari, Haryana (IN); Ram Chander Aryan, Delhi (IN); Bishwa Prakesh Rai, Uttar Pradesh (IN); Hashim Nizar, Kerala (IN)

Correspondence Address: RANBAXY INC., 600 COLLEGE ROAD EAST, SUITE 2100, PRINCETON, NJ, 08540, US

(21) Appl. No.: 10/475,276

(22) PCT Filed: Apr. 17, 2002
(86) PCT No.: PCT/IB02/01240

(30) Foreign Application Priority Data
Apr. 17, 2001 (IN) 493/DEL/2001

(51) Int. Cl. A61K 31/545; C07D 501/14

(52) U.S. Cl. 514/202; 540/222

(57) ABSTRACT

The present invention relates to an improved and cost effective process for the industrial preparation of cefpodoxime acid of Formula (I) and a pharmaceutically acceptable ester thereof.

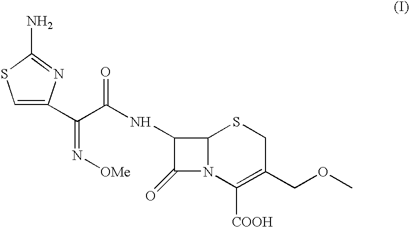

### FIELD OF THE INVENTION

The present invention relates to an improved and cost effective process for the industrial preparation of cefpodoxime acid of Formula I 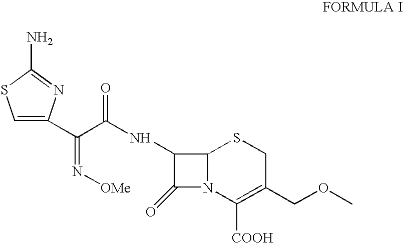
and a pharmaceutically acceptable ester thereof.

### BACKGROUND OF THE INVENTION

Chemically, cefpodoxime acid is [(6R-[6α,7β(Z)]]-7-[2-(2-aminothiazol-4-yl)-2-methoxyimino) acetamido]-3-cephem-4-carboxylic acid having Formula I, and is known from U.S. Pat. No. 4,409,215. Although cefpodoxime acid is not suitable for oral administration, its ester derivative, 1-(isoproxycarbonyloxyl)ethyl ester i.e. cefpodoxime proxetil of Formula II, 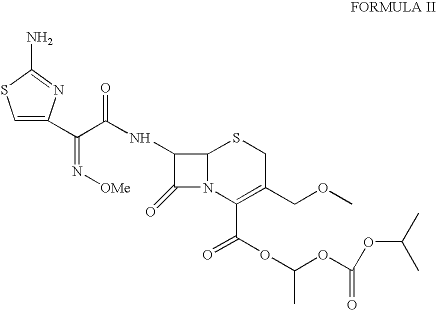
is a valuable orally administered antibiotic characterized by high broad spectrum activity against gram positive and gram negative microorganisms.

A number of processes have been outlined in U.S. Pat. Nos. 4,409,215, 5,109,131, GB 2012276 and WO 00/63214 for the preparation of cepholosporin antibiotics. However, attempts to extend these processes for preparing cefpodoxime acid at an industrial scale did not give the desired results with respect to yield and quality. More particularly, the synthetic process comprising coupling of reactive acid derivative of compound of Formula III, 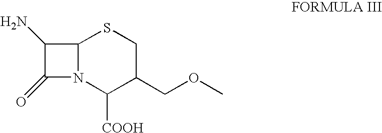
with a reactive derivative of an open chain compound of Formula IV, 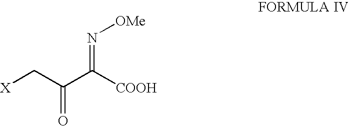
wherein X is a halogen selected from chloro, bromo and iodo, to get a compound of Formula V, 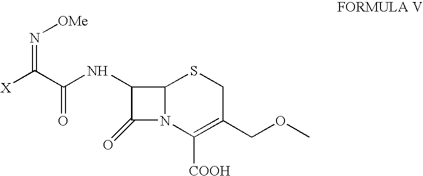
and its subsequent cyclization with thiourea to obtain cefpodoxime acid of Formula I, was found to be unsatisfactory at a commercial scale. Processes described in U.S. Pat. No. 4,409,215 and GB 2012276 require protection at the carboxylic position of the compound of Formula III followed by the steps of coupling, cyclization and hydrolysis to get cefpodoxime acid. The additional steps of protection and deprotection result in low yields and high costs. The processes described in PCT Application WO 00/63214 and U.S. Pat. No. 5,109,131 require formation of compound of Formula V and its subsequent cyclization with thiourea in a mixture of organic solvent and water to afford cefpodoxime acid. Cefpodoxime acid thus obtained is of poor quality and contains anti isomer of cefpodoxime acid as a major impurity.

Accordingly, none of the processes described heretofore are completely satisfactory for various reasons.

### SUMMARY OF THE INVENTION

It is an object of the present invention to provide a process for the preparation of cefpodoxime acid and a pharmaceutically acceptable ester thereof in good yields and high purity (99%) by HPLC. The process is simple and provides obvious benefits with respect to economics and convenience to operate at a commercial scale.

Accordingly, the present invention provides a process for the preparation of cefpodoxime acid of Formula I 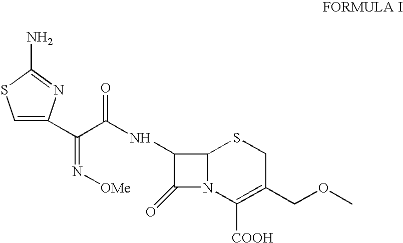
and a pharmaceutically acceptable ester thereof comprising:
(i) reacting a compound of Formula VI, 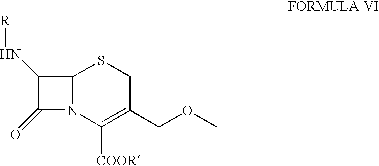
   wherein R is hydrogen or a silyl group and R′ is a silyl group or COOR′ is a carboxylic acid salt, with a compound of Formula IV, 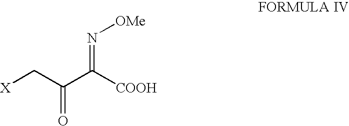
   or its reactive acid derivatives, wherein X is a halogen, to obtain a compound of Formula VII, 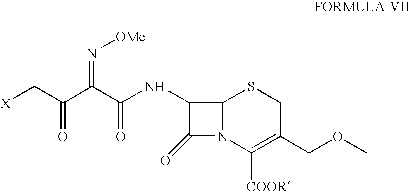
   wherein X and R′ are as defined above;
(ii) desilylating or acidifying the compound of Formula VII to isolate the compound of formula V; and 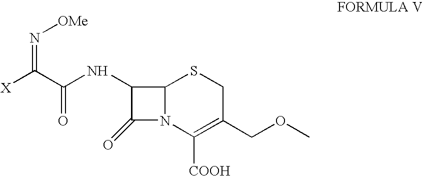
(iii) reacting the compound of Formula V with thiourea in aqueous medium in the presence of a weak base to obtain cefpodoxime acid of Formula I.

Cefpodoxime acid, so obtained may be converted into its ester such as cefpodoxime proxetil by methods known in the art.

The carboxylic acid salts of Formula VI include salts with a metal such as sodium or potassium, or salt with an organic amine such as triethylamine, pyridine, diclyclohexylamine or N, N-dimethylaniline.

R and R′ in the compound of Formula VI may be silyl groups which may be same or different. Suitable silyl groups are trialkyl silyl groups wherein the alkyl substitutents may be same or different. Preferred alkyl substituents are methyl, ethyl, isopropyl and tert-butyl. Preferred silyl groups are trimethylsilyl and tert-butyidimethylsilyl.

X in the compounds of Formula IV, V and VIII is a halogen selected from chloro, bromo and iodo. X is preferably bromo.

The reactive acid derivatives of Formula IV include the acid halides, the acid anhydride, mixed acid anhydrides, reactive esters, reactive amides and the acid azide. Preferred mixed acid anhydrides include anhydrides with lower alkanoic acids such as pivalic acid, trichloroacetic acid and anhydrides with a carbonic acid such as monomethylcarbonate. Preferred reactive esters include p-nitrophenylester, N-hydroxysuccinimido ester, N-hydroxyphthalimido ester, 2-mercaptobenzothioazolyl ester and 2-mercapto-5-methyl-1,3,4-thiadiazolyl ester. Among the reactive acid derivatives of Formula IV, acid halides are preferred.

Where the compound of Formula IV is employed in the form of a free acid, the reaction step (i) is carried out in the presence of a condensing agent such as dicylohexylcarbodiimide, or a “Vilsmeier reagent” formed by the reaction of an amide compound such as dimethylformamide with a halogen compound such as phosphorous oxychloride.

Where a reactive derivative of the acid of Formula IV is employed, the use of such a condensing agent is not required, however, it may be desirable to carry out the reaction in the presence of a base. Examples of suitable bases include alkali metal compound such as sodium bicarbonate, sodium carbonate and potassium carbonate or an organic amine such as triethylamine, lutidine or pyridine.

The reaction of step (i) is usually carried out in a suitable solvent. When R, R′ or both are silyl in the compound of Formula VI, suitable solvents for the reaction include halogenated hydrocarbons such as methylene chloride, hydrocarbons such as toluene, ethers such as tetrahydrofuran or polar solvents such as dimethylformamide, or a mixture thereof. When R is hydrogen and COOR′ is a carboxylic acid salt in the compound of Formula VI, suitable solvents for the reaction include methanol, ethanol, acetonitrile, dimethylformamide, water, or a mixture thereof.

The starting compounds of Formula VI wherein R, R′ or both are silyl may be obtained by silylating the corresponding 7-amino-3-methoxymethyl 3-cephem-4-carboxylic acid of Formula III with a suitable silylating agent. Appropriate silylating agents include halosilanes such as trimethylsilylchloride (TMCS), dimethyidichlorosilane (DMDCS), silylated amides such as N, 0-bistrimethylsilyl acetamide (BSA), silazanes such as 1,1,1,3,3,3-hexamethyidisilazane (HMDS), silylated ureas such as N, N′-bis-(trimethylsilyl) urea (BSU), or a mixture thereof

Where COOR′ is a carboxylic acid salt in the compound of Formula VI, it may be obtained in a conventional manner, for example by treatment of a compound of Formula III with a base such as sodium bicarbonate, triethylamine etc.

Compounds of Formula III and IV may be obtained by methods known in the art.

The desilylation (step ii) of the compound of Formula VII (wherein R′ is a silyl group) may be carried out according to conventional methods such as treatment with methanol/water to isolate compound of Formula V.

We believe that the isolation of the compound of Formula V plays a crucial role in obtaining the compound of Formula I in high yields and good quality. The reactions of steps (i) and (ii) result in the formation of impurities alongwith the desired product which are automatically removed during the isolation of compound of Formula V.

The reaction of a compound of Formula V with thiourea is carried out in the presence of a weak base such as sodium acetate and sodium bicarbonate in an aqueous medium comprising water and a water-miscible organic solvent such as ethanol, methanol, isopropanol, acetone, tetrahydrofuran, acetonitrile, N, N-dimethylformamide, or a mixture thereof. The compound of Formula V is added to an aqueous solution of a weak base at a temperature of about 0 to 5° C. Thereafter, an aqueous solution of thiourea is added to the above mixture at a temperature of about 0 to 10° C. The reaction may then be carried out a temperature of about 0to 60° C., preferably at 0-25° C., more preferably at 10-20° C. Cefpodoxime acid of purity 99% is obtained by washing the reaction mixture with ethyl acetate and acidifying the aqueous layer to a pH of about 2.5 to 3.

However, the reaction of compound of Formula V with thiourea is best carried out in water since a mixture of solvent and water may carryover impurities to the aqueous layer which may then precipitate along with the desired product. Also, lower yields are obtained as cefpodoxime acid is soluble in the water-miscible solvents mentioned above.

Cefpodoxime acid so obtained may be converted to cefpodoxime proxetil by methods known in the art such as reaction with 1-iodoethylisopropyl carbonate in the presence of 1,8-diazabicyclo [5.4.0] undec-7-ene (DBU) in N, N-dimethylformamide.

### DETAILED DESCRIPTION OF THE INVENTION

In the following section a preferred embodiment is described by way of example to illustrate the process of this invention. However, it is not intended in any way to limit the scope of the present invention.

### EXAMPLE

Preparation of Cefpodoxime Acid

(i) 7-[4-bromo-3-oxo-(Z)-2-methoxyiminobutyrylamino]-3-methoxymethyl-3-cephem-4-carboxylic acid

Solution A

Hexamethyidisilazane (73.9 g) and acetamide (54.2 g) were refluxed in dichloromethane (560 ml) in the presence of a catalytic amount of imidazole. 7-amino-3-methoxymethyl-3-cephem-4-carboxylic acid (80.0 g) was added to the resulting solution and refluxed for 1 hour to obtain almost a clear solution.

Solution B

Phosphorous pentachloride (66.2 g) was added to a solution of 4-bromo-2-methoxyimino-3-oxobutyric acid (69.8 g) in dichloromethane at about −20 to −10° C. and stirred for about one hour.

Solution A was added to solution B at about −70 to −50° C. and further stirred at about −30 to −10° C. for two hours. The reaction mixture was then poured into a mixture of water and methanol. The organic layer was separated, concentrated to about 240 ml and toluene (800 ml) was added to the concentrated mass to obtain the title compound (110 g) after filtration and drying at 30° C.

(ii) 7-[2-(aminothiazol-4-yl)-2-(Z)-methoxyiminoacetamido]-3-methoxymethyl-3-cephem-4-carboxylic acid

7-[4-bromo-3-oxo-(Z)-2-methoxyiminobutyrylamino]-3-methoxymethyl-3-cephem-4-carboxylic acid (90 g)obtained from step (i) was added to a cold (2-5° C.) solution of sodium acetate (163.2 g) in water (720 ml). Thereafter, a solution of thiourea (18.3 g) in water was added to it at 0-10° C. The mixture was stirred at 15-20° C. for about two hours. The reaction mixture was then washed with ethyl acetate and pH of the aqueous layer was adjusted to about 2.5-3 to obtain cefpodoxime acid (70 g; purity by HPLC=99%) after filtration and drying at 45-50° C.

Preparation of Cefpodoxime Proxetil

1-isopropoxycarbonyloxyethyl-7-[2-(2-aminothiazol-4-yl)-2-(Z)-methoxyiminoacetamido]-3-methoxymethyl-3-cephem-4-carboxylate

7-[2-(2-aminothiazol-4-yl)-2-(Z)-methoxyiminoacetamido]-3-methoxymethyl-3-cephem-4-carboxylic acid (50 g) was dissolved in N, N-dimethylacetamide (300 ml) and to this solution was added 1,8-diazabicyclo[5,4,0]undec-7-ene (DBU) (17.11 g) at −10 to 0° C. Iodoethylisopropyl carbonate (30.19 g) was then added to the resulting mixture at the same temperature. The reaction was worked up after stirring for two hours at −10 to −5° C. by addition of ethyl acetate and water. The organic layer was separated and successively washed with 0.2% aqueous hydrochloric acid solution, 1% aqueous sodium bicarbonate solution and finally 1% aqueous sodium thiosulfate solution.

The organic layer was concentrated to about 200 ml and the product precipitated with cyclohexane (1500 ml). The product so obtained was purified by reprecipitation with methanol/water to obtain pure cefpodoxime proxetil (48 g; purity by HPLC=98%).

While this invention has been described by reference to specific examples, this was for the purpose of illustration only. Numerous alternative embodiments will be apparent to those skilled in the art and are considered to be within the scope of this invention.

1. A process for the preparation of cefpodoxime acid of Formula I, 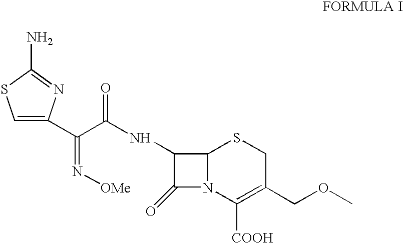
and a pharmaceutically acceptable ester thereof, comprising
(i) reacting a compound of Formula VI, 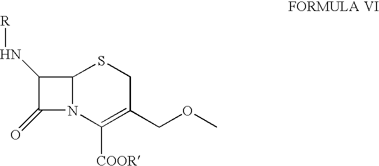
wherein R is hydrogen or a silyl group and R′ is a silyl group or COOR′ is a carboxylic acid salt,
with a compound of Formula IV, 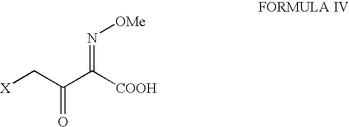
or its reactive acid derivatives, wherein X is a halogen, to obtain a compound of Formula VII, 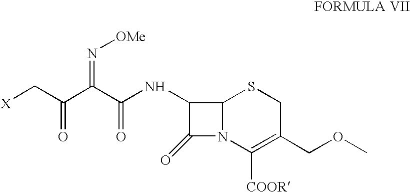
wherein X and R′ are as defined above;
(ii) desilylating or acidifying the compound of Formula VII to isolate the compound of formula V; and 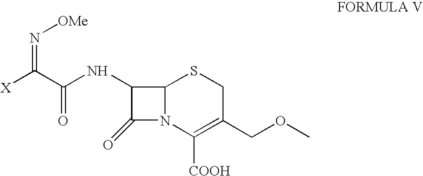
(iii) reacting the compound of Formula V with thiourea in aqueous medium in the presence of a weak base to obtain cefpodoxime acid of Formula I.

2. The process according to claim 1 wherein both R and R′ are trimethylsilyl in the compound of Formula VI.

3. The process according to claim 1 wherein X is chloro or bromo in the compound of Formula IV.

4. The process according to claim 1 wherein the reactive derivative of Formula IV is acid chloride.

5. The process according to claim 1 wherein the reaction of step (iii) in aqueous medium comprises reacting in water and a water-miscible organic solvent.

6. The process according to claim 5 wherein the water-miscible organic solvent is selected from the group consisting of ethanol, methanol, isopropanol, acetone, tetrahydrofuran, acetonitrile, N,N-dimethylformamide, or a mixture thereof.

7. The process according to claim 5 wherein the reaction of step (iii) is carried out in water alone.

8. The process accounting to claim 1 wherein the weak base in step (iii) is selected from the group consisting of sodium acetate or sodium bicarbonate.

9. The process according to claim 1 wherein in step (iii), the compound of Formula V is added to an aqueous solution of sodium acetate or sodium bicarbonate at a temperature of about 0 to 50° C.

10. The process according to claim 1 wherein in step (iii), the thiourea is added at a temperature of about 0 to 10° C.

11. The process according to claim 1 wherein the reaction of step (iii) is carried out at a temperature of about 10 to 20° C.

12. The process according to claim 1 wherein the cefpodoxime acid is obtained at a pH of about 2.5 to 3.0.

13. The process according to claim 1 wherein the cefpodoxime acid of Formula I is reacted with 1 -iodoethylisopropyl carbonate in the presence of 1,8-diazabicyclo[5,4,0]undec-7-ene (DBU) in N,N-dimethylformamide to give cefpodoxime proxetil of Formula II [chemical structure]
